(19) United States
(12) Patent Application Publication
(10) Pub. No.: US 2004/0243184 A1
(43) Pub. Date: Dec. 2, 2004

(54) External defibrillator powered by fuel cell

(76) Inventors: Stephen B. Johnson, Clinton, WA (US); Patrick F. Kelly, Edmonds, WA (US)

Correspondence Address: SHUMAKER & SIEFFERT, P. A., 8425 SEASONS PARKWAY, SUITE 105, ST. PAUL, MN, 55125, US

(21) Appl. No.: 10/448,744

(22) Filed: May 30, 2003

(51) Int. Cl. A61N 1/39

(52) U.S. Cl. 607/5

(57) ABSTRACT

The invention is directed to external defibrillators that are powered by fuel cells. A fuel cell provides a voltage to power components of a defibrillator, such as a processor and a user interface, and to charge an energy storage circuit, e.g., a capacitor, that stores energy for delivery to a patient as a defibrillation shock. A user may use an activator to activate the fuel cell. In some embodiments, the activator includes a button that a user actuates to cause delivery of fuel to the fuel cell.

### TECHNICAL FIELD

The invention relates to medical devices and, more particularly, to power sources for external defibrillators.

### BACKGROUND

Cardiac arrest and ventricular fibrillation are life threatening medical conditions that may be treated with external defibrillation. External defibrillation involves applying electrodes to the chest of a patient, and delivering an electric shock via the electrodes to depolarize the heart of the patient and restore normal sinus rhythm. External defibrillators that provide electric shocks for defibrillation are used in hospitals, and by paramedics, emergency medical technicians, police officers, and the like to respond to medical emergencies in the field. Additionally, automated external defibrillators (AEDs) are often located in public venues, such as airports, health clubs and auditoriums, to allow minimally trained operators to deliver prompt external defibrillation in response to a medical emergency.

Before an external defibrillator is used to administer a shock, the energy to be delivered to the patient must be stored in an energy storage device, such as a capacitor. Defibrillators typically use a charging circuit to transfer energy from a power source, such as a battery, to the energy storage device. When a switch is closed, the energy storage device delivers at least a part of the stored energy across the electrodes and through the patient's chest.

External defibrillators typically use one or more rechargeable, chemical batteries, such as nickel-cadmium batteries, sealed lead acid batteries or nickel-metal-hydride batteries, as a power source. Some rechargeable batteries have a short shelf life. Nickel-metal-hydride batteries, for example, discharge within a few months, even when no load is applied. Further, some rechargeable batteries, such as nickel-cadmium batteries, need to undergo conditioning cycles periodically to deliver optimum performance.

Establishing and overseeing a defibrillator maintenance program can be a significant administrative burden, particularly for large hospitals, EMS systems, and public facilities. Because each recharging or conditioning of the batteries of a defibrillator takes a significant amount of time, the cost of the skilled labor required to maintain external defibrillators can be quite high. Further, there is the possibility that defibrillators will not be adequately maintained, leaving those defibrillators unable to provide defibrillation therapy when needed. Inadequate maintenance is a particular problem with AEDs, which are ordinarily installed at a location within a public facility, and sometimes forgotten until they are needed to respond to emergency that may not occur for months or even years after installation.

### SUMMARY

The invention is directed to an external defibrillator that is powered by a fuel cell. A fuel cell provides energy to power components of a defibrillator, such as a processor and a user interface, or to charge an energy storage circuit, such as a capacitor, that stores energy for delivery to a patient as a defibrillation shock. A user may use an activator to activate the fuel cell. In some embodiments, the activator includes a button that a user actuates to cause delivery of fuel to the fuel cell.

In some embodiments, the defibrillator includes a secondary power source, which may be a second fuel cell or a battery, that power components of the defibrillator when it is not in use, e.g. when the primary fuel cell is inactive. The secondary power source may provide power to allow the defibrillator to perform self-check routines, and indicate status, e.g., readiness to provide defibrillation therapy, to users.

In one embodiment, the invention is directed to an external defibrillator that includes an energy storage circuit to store energy for delivery to a patient as a defibrillation shock, and a fuel cell coupled to the energy storage circuit to provide energy to charge the energy storage circuit for delivery of the defibrillation shock. The external defibrillator further includes electrodes that are selectively coupled to the energy storage circuit by a switch to deliver the defibrillation shock to the patient. The energy storage circuit may include a capacitor, and the external defibrillator may be an automatic external defibrillator.

In another embodiment, the invention is directed to an external defibrillator that includes an energy storage circuit to store energy for delivery to a patient as a defibrillation shock, a fuel cell coupled to the energy storage circuit to provide energy to charge the energy storage circuit for delivery of the defibrillation shock, and electrodes that are selectively coupled to the energy storage circuit by a switch to deliver the defibrillation shock to the patient. The external defibrillator further includes an activator that allows a user to activate the fuel cell. The activator may include a button, and the user may press the button to activate the fuel cell. The defibrillator may include a cover, and the user may press the button to open the cover and activate the fuel cell. The activator may enable delivery of hydrogen to the fuel cell.

In another embodiment, the invention is directed to an external defibrillator that includes an energy storage circuit to store energy for delivery to a patient as a defibrillation shock, a fuel cell coupled to the energy storage circuit to provide energy to charge the energy storage circuit for delivery of the defibrillation shock, electrodes that are selectively coupled to the energy storage circuit by a switch to deliver the defibrillation shock to the patient, and an activator that allows a user to activate the fuel cell. The external defibrillator further includes a processor and a user interface that are powered by the fuel cell when the fuel cell is activated, and by a secondary power source when the fuel cell is not activated. The secondary power source may be another fuel cell or a battery. The processor may perform a self-test during a period when the fuel cell is not activated to evaluate readiness of the defibrillator to deliver therapy, and provides an indication of readiness to a user via the user interface.

In another embodiment, the invention is directed to a method of powering an external defibrillator in which energy from a fuel cell is delivered to components of the defibrillator. Fuel may be delivered to the to the fuel cell, and energy may be delivered from the fuel cell to the components as a function of the delivery of fuel to the fuel cell. An activator may be actuated to cause delivery of fuel to the fuel cell.

In another embodiment, the invention is directed to a method of operating a defibrillator in which an activator is actuated to activate a fuel cell and power on the defibrillator. Actuating an activator may comprise pressing a button of the defibrillator. Actuating an activator may also comprise opening a lid of the defibrillator.

The invention may provide one or more advantages. For example, unlike conventional defibrillator batteries, fuel cells do not require conditioning, and their disposal may not raise the environmental concerns associated with conventional defibrillator batteries. Also, because of the energy storage density of the fuel used by fuel cells and their efficiency, fuel cells may not need to be replenished as often as conventional defibrillator batteries need to be recharged. Consequently, use of fuel cells to power external defibrillators may reduce the burden associated with maintaining external defibrillators.

Further, unlike conventional defibrillator batteries, fuel cells can be configured to remain substantially inactive, i.e., configured so that fuel is not delivered to the fuel cell, when not in use. Because fuel cells may be configured so that they do not lose their “charge” when not in use, the frequency of recharging may be further reduced when compared to conventional defibrillator batteries. Further, the ability of a fuel cell-powered defibrillator to remain charged, i.e., in a state of readiness to provided defibrillation therapy, for a substantially unlimited period of time when not used may be particularly desirable in the case of infrequently used and potentially neglected AEDs.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1A is a perspective diagram illustrating an example external defibrillator powered by a fuel cell according to an embodiment of the invention.

FIG. 1B is a perspective diagram illustrating an example base for the external defibrillator of FIG. 1A according to an embodiment of the invention.

FIG. 2 is a conceptual diagram illustrating a fuel cell module for use in an external defibrillator according to an embodiment of the invention.

FIG. 3 is a block diagram illustrating components of the example external defibrillator of FIG. 1 according to an embodiment of the invention.

FIG. 4 is a flowchart illustrating example operation of the external defibrillator of FIG. 3 according to an embodiment of the invention.

FIG. 5 is a block diagram illustrating components of an example external defibrillator that includes a fuel cell and a secondary power source according to an embodiment of the invention.

FIG. 6 is a flowchart illustrating example operation of the external defibrillator of FIG. 5 according to an embodiment of the invention.

### DETAILED DESCRIPTION

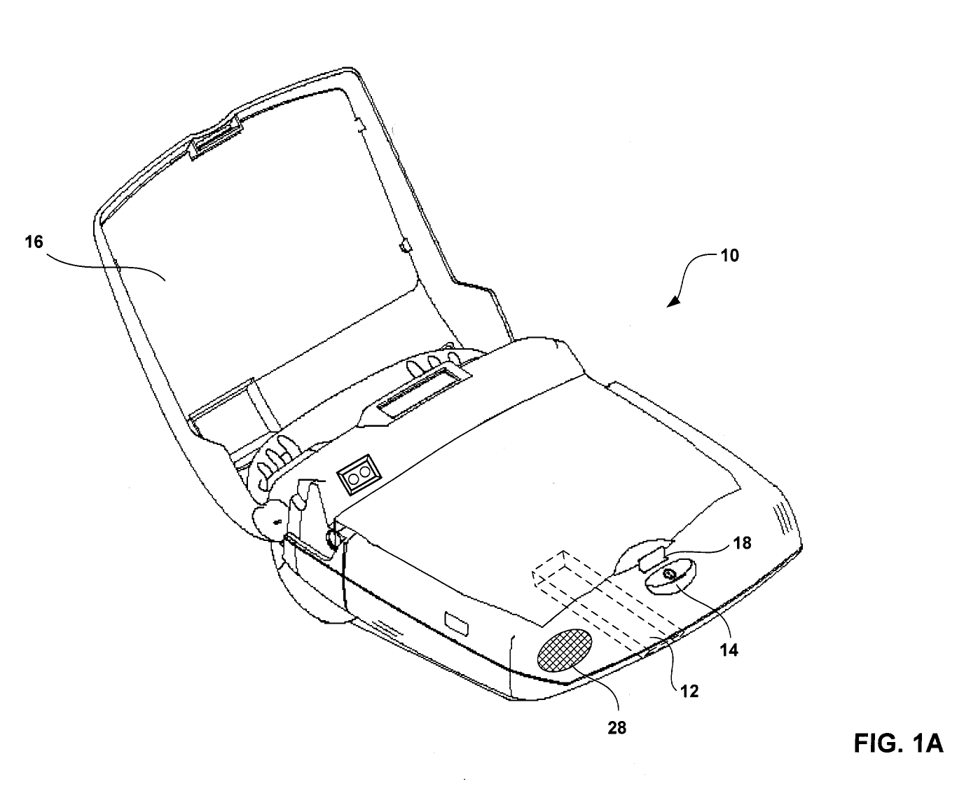

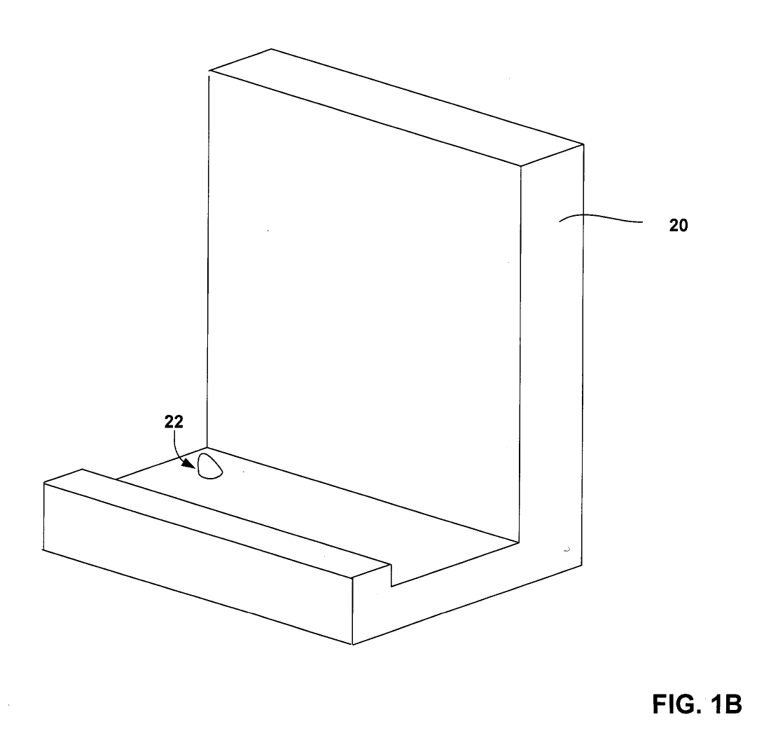

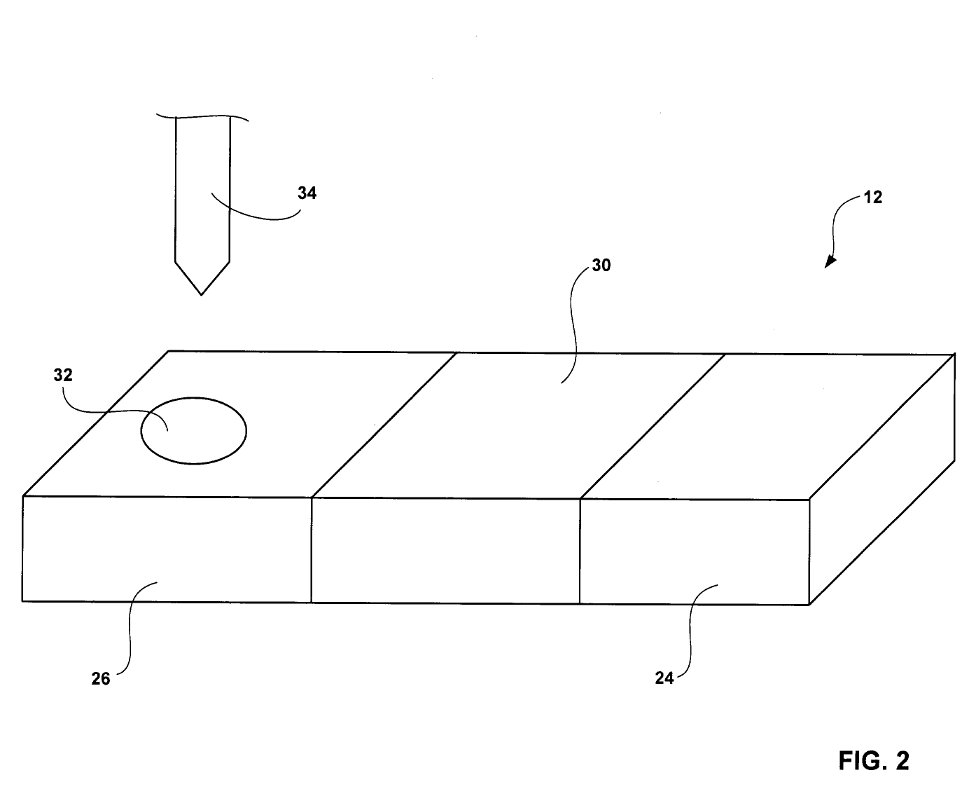

FIG. 1A is a perspective diagram illustrating an example external defibrillator 10 that is powered by a fuel cell. The fuel cell may a component of a fuel cell module 12, as will be described in greater detail below with reference to FIG. 2. Defibrillator 10 may take the form of a clinical or portable defibrillator/monitor, or, as shown in FIG. 1A, an automatic external defibrillator (AED).

The fuel cell provides energy that is used by defibrillator 10 to deliver electric shocks to patients for defibrillation. The fuel cell also may provide energy that is used to power a microprocessor (not shown), a user interface (not shown), and other components of defibrillator 10. Other systems that may be included as part of defibrillator 10, such as communication and patient monitoring systems, also may be powered by the fuel cell.

As discussed above, the rechargeable batteries typically used in conventional defibrillators lose charge over time, even when no load is applied, and, in some cases, must be periodically conditioned to operate properly. Consequently, conventional defibrillators require time-consuming maintenance, even when they are not used. Further, the batteries used in conventional defibrillators are chemical batteries, which often require special handling when disposed at the end of their useful life due to environmental concerns. Even though they are rechargeable, conventional defibrillator batteries may need to be replaced a number of times during the serviceable life of a defibrillator.

Fuel cells do not require conditioning, and, because they do not need to be disposed of until the associated defibrillator is disposed, their use may have a lesser environmental impact then the use of conventional defibrillator batteries. Also, because of the energy storage density of the fuel used by fuel cells and their efficiency, fuel cells may not need to be recharged as often as conventional defibrillator batteries. Consequently, use of a fuel cell to power defibrillator 10 may reduce the burden associated with maintaining defibrillator 10.

Unlike conventional defibrillator batteries, fuel cells can be configured to remain substantially inactive, i.e., configured so that fuel is not delivered to the fuel cell, when not in use. Because fuel cells may be configured so that they do not lose their “charge” when not in use, the frequency of recharging may be further reduced when compared to conventional defibrillator batteries. Further, the ability of fuel cell powered defibrillator 10 to remain charged, i.e., in a state of readiness to provided defibrillation therapy, for a substantially unlimited period of time when not used may be particularly desirable in cases where defibrillator 10 takes the form of an infrequently used and potentially neglected AED.

The fuel cell of defibrillator 10 may be activated, i.e., the delivery of fuel to the fuel cell may be initiated, in any of a number of ways, and the invention is not limited to any particular technique or mechanism for fuel cell activation. As one example, an activator for activating the fuel cell may include a button 14 on the housing of defibrillator 10. The activator may include additional electrical components (not shown), e.g., switches and circuits, and/or mechanical components (not shown) that enable delivery of fuel to the fuel cell upon actuation of button 14. In some embodiments, button 14 may take the form of a mechanical switch or a soft-key.

In general, activation of the fuel cell should occur at a time when a user would expect defibrillator 10 to be powered on. To that end, button 14 may act, and be labeled, as a “power-on” button for defibrillator 10. In the exemplary embodiment illustrated in FIG. 1A, defibrillator 10 includes a cover 16 that a user opens to expose electrodes (not shown), a display (not shown), and other buttons, keys, switches, or the like (not shown) that facilitate provision of defibrillation therapy to a patient using defibrillator 10. In such an embodiment, in addition to activating the fuel cell, actuation of button 14 by a user may release a latch 18 to allow lid 16 to open. Thus, when a user begins to use defibrillator 10 to treat a patient by actuating button 14 to open lid 16, components of an activator coupled to button 14 will initiate delivery of fuel to the fuel cell to power on defibrillator 10.

The invention is not, however, limited to the example illustrated in FIG. 1A. For example, button 14 may be separate from a button used to open lid 16, or defibrillator 10 may not include a lid 16. Further, an activator for activating a fuel cell need not include button 14 at all. For example, lid 16 may be coupled or otherwise interact with electrical or mechanical components of the activator such that the mechanical motion associated with opening lid 16 causes delivery of fuel to the fuel cell. In such an embodiment, lid 16 may be coupled to or interact with, for example, a reed switch that is in turn coupled to a circuit such that when the lid is open a pump that delivers fuel to the fuel cell is activated.

When not being used to treat a patient, defibrillator 10 may be situated on a base 20, shown in FIG. 1B. Base 20 may provide support for defibrillator 10 such that defibrillator 10 may be mounted on a wall, or the like. In embodiments where defibrillator 10 is mounted on base 20, defibrillator 10 may be configured such that removal of defibrillator 10 from base 20, e.g., by a user wishing to use defibrillator 10 to provide defibrillator therapy to a patient, activates the fuel cell and powers on defibrillator 10.

Base 20 may, as shown in FIG. 1B, include a protrusion 22. Protrusion 22 may be positioned on base 20, and button 14 (FIG. 1) may be positioned on defibrillator 10, such that protrusion 22 depresses button 14 when defibrillator 10 is situated on base 20. In such embodiments, the fuel cell of defibrillator 10 may be activated by removal of defibrillator 10 from base 20 such that protrusion 22 no longer depresses button 14. Additional electrical components (not shown), e.g., switches and circuits, and/or mechanical components (not shown) may be coupled to button 14 to enable delivery of fuel to the fuel cell upon release of button 14.

The configuration of base 20 illustrated in FIG. 1B is merely exemplary. In some embodiments, base 20 may take the form of a mounting bracket. In other embodiments, defibrillator 10 may not be mounted on a vertical structure. In some embodiments, base 20 may include a case with a door or breakable glass pane to allow access to defibrillator 10.

FIG. 2 is a conceptual diagram illustrating an example fuel cell module 12 according to an embodiment of the invention. As shown in FIG. 2, fuel cell module 12 includes a fuel cell 24 and a container 26 to store fuel for fuel cell 24. Fuel cell 24 may correspond to any of a number of known types of fuel cells, and the invention is not limited to any particular type of fuel cell. A description of exemplary fuel cell types is provided by Haile, Sossina M., “Swiss Rolls and Oreo Cookies,” Engineering and Science, Vol. LXVI, No. 1, California Institute of Technology, 2003 (hereinafter “Haile”), which is incorporated by reference herein in its entirety.

Fuel cell 24 generates a voltage between an anode and a cathode to power defibrillator 10 as a function of the reaction of hydrogen and oxygen to create water. Fuel cell 24 may receive oxygen for the reaction from air, and release water vapor resulting from the reaction into the air. Defibrillator 10 may include a vent 28 (FIG. 1A) to allow the air surrounding defibrillator 10 to enter the housing of defibrillator 10 and interact with fuel cell 24. Defibrillator 10 may include water collection, evaporation, or wicking mechanisms to handle the water byproduct of the generation of energy by fuel cell 24.

The fuel within container 26 is the source of hydrogen for generation of energy by fuel cell 24. Exemplary fuels that may be used as a source of hydrogen for fuel cell 24 include alcohol, methanol, propane, and butane. In the embodiment illustrated in FIG. 2, fuel cell module 12 includes a reformer 30 to extract hydrogen from one or more of the above-identified fuels, and provide the hydrogen to fuel cell 24.

FIG. 2 illustrates an exemplary mechanism for delivering a liquid fuel, such as alcohol, methanol, or butane, from container 26 to reformer 30. Container 26 may include a membrane 32 that is pierceable by a puncture member 34. Puncture member 34 is a component of an activator for activating fuel cell 24, i.e., initiating delivery of fuel to reformer 30.

Puncture member 34 may be mechanically coupled to an actuator operated by a user. For example, puncture member 34 may be coupled to button 14 (FIG. 1A), such that actuation of button 14 causes puncture member 34 to descend and pierce membrane 32. Where defibrillator 10 is situated on a base 20 with a protrusion 22 that depresses button 14, as described above with reference to FIG. 1B, puncture member 34 may be coupled to button 14 such that removal of defibrillator 10 from base 20 causes puncture member 34 to descend and pierce membrane 32. A liquid fuel may be stored in container 26 under a vacuum, such that the surface tension of the fuel keeps the fuel from entering the reformer until membrane 32 is pierced by puncture member 34.

The invention is not, however, limited to illustrated container 26 and associated delivery techniques, or to use of liquid fuels. In some embodiments, container 26 may include a valve that is opened by the activator to allow a liquid or gaseous fuel to flow to reformer 30. The valve may be metered, and may be controlled to open and close by an activator to allow defibrillator 10 to be used multiple times without refueling.

In some embodiments, fuel cell 24 may be a “direct fuel” fuel cell, such as a direct methanol fuel cell. In other embodiments, container 26 may simply contain hydrogen for delivery to fuel cell 24. In such embodiments, fuel cell module 12 need not include reformer 30.

To recharge fuel cell 24, container 26 is refilled. In some embodiments, container 26 may be removed, and either replaced with a new, full container 26, or refilled and replaced. In other embodiments, container 26 may include a valve or port that is accessible from the exterior of defibrillator 10 for refilling.

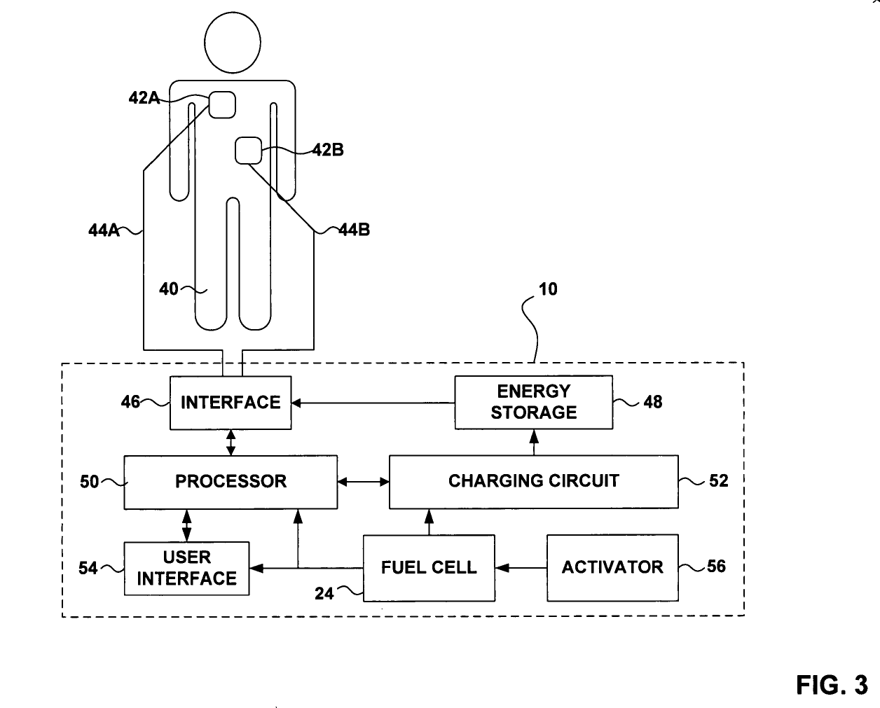

FIG. 3 is a block diagram illustrating components of external defibrillator 10 according to an embodiment of the invention. Defibrillator 10 is shown in FIG. 3 coupled to a patient 40 via electrodes 42A and 42B (collectively “electrodes 42”). Electrodes 42 may be hand-held electrode paddles or adhesive electrode pads placed on the skin of patient 40. Electrodes 42A and 42B are coupled to defibrillator 10 by conductors 44A and 44B (collectively “conductors 44”), respectively.

Conductors 44 are coupled to an interface 46. In a typical application, interface 46 includes a receptacle, and conductors 44 plug into the receptacle. Interface 46 may also include a switch that, when activated, couples an energy storage circuit 48 to electrodes 42.

Energy storage circuit 48 includes components, such as one or more capacitors, which store the energy to be delivered to patient 40 via electrodes 42 as a defibrillation shock. Before a defibrillation shock may be delivered to patient 40, energy storage circuit 48 must be charged. A processor 50 directs a charging circuit 52 to charge energy storage circuit 48 to a voltage level determined by processor 50. Processor 50 may determine the voltage level based on a defibrillation shock energy level that may be, for example, input by a user via user interface 54, or selected by processor 50 from a preprogrammed progression of defibrillation shock energy levels stored in a memory (not shown).

Processor may activate the switch within interface 46 to cause delivery of the energy stored in energy storage circuit across electrodes 44. Processor 50 may modulate the defibrillation shock delivered to patient 40. Processor 50 may, for example, control the switch to regulate the shape of the waveform of the shock and the width of the shock. Processor 50 may control the switch to modulate the shock to, for example, provide a multiphasic pulse, such as a biphasic truncated exponential pulse, as is known in the art. Processor 50 may take the form of a microprocessor, digital signal processor (DSP), application specific integrated circuit (ASIC), field-programmable gate array (FPGA), or other logic circuitry programmed or otherwise configured to operate as described herein.

User interface 54 may include a display. Processor 50 may display instructions to a user via the display, and an electrocardiogram (ECG) and heart rate of patient 40 monitored via electrodes 42 may also be displayed via the display. Defibrillator 10 may include circuits (not shown) known in the art for monitoring a variety of physiological parameters of patient 40, such as blood pressure and blood oxygen saturation, and the display may be used to display the values for these parameters measured by the circuits. User interface 54 may also include various buttons, soft-keys, knobs, switches, or the like used by a user to control the operation of defibrillator 10.

When activated by activator 56, as described above, fuel cell 20 generates energy to power processor 50 and, for those components that require power, user interface 54. Activator 56 may, as described above, include button 14 (FIG. 1) coupled to puncture member 30, such that, when button 14 is actuated, puncture member 30 pierces membrane 28 to allow fuel to flow from container 22 to one of reformer 26 or fuel cell 20. Under the control of processor 50, charging circuit 52 transfers energy provided by fuel cell 20 to energy storage circuit 48 for delivery as a defibrillation shock to patient 40. Charging circuit 52 comprises, for example, a flyback charger.

In addition to providing power for defibrillation shocks, and for microprocessor 50 and user interface 54, fuel cell 20 may provide power for other components of defibrillator 10 not illustrated in FIG. 3, such as the physiological monitoring circuits and memory described above. Although described herein as a single fuel cell, it is understood that fuel cell 20 may comprise a number of fuel cells arranged in series to provide a desired voltage. Moreover, it is understood that the voltage provided by fuel cell 20 may be regulated as necessary for use by the components of defibrillator 10.

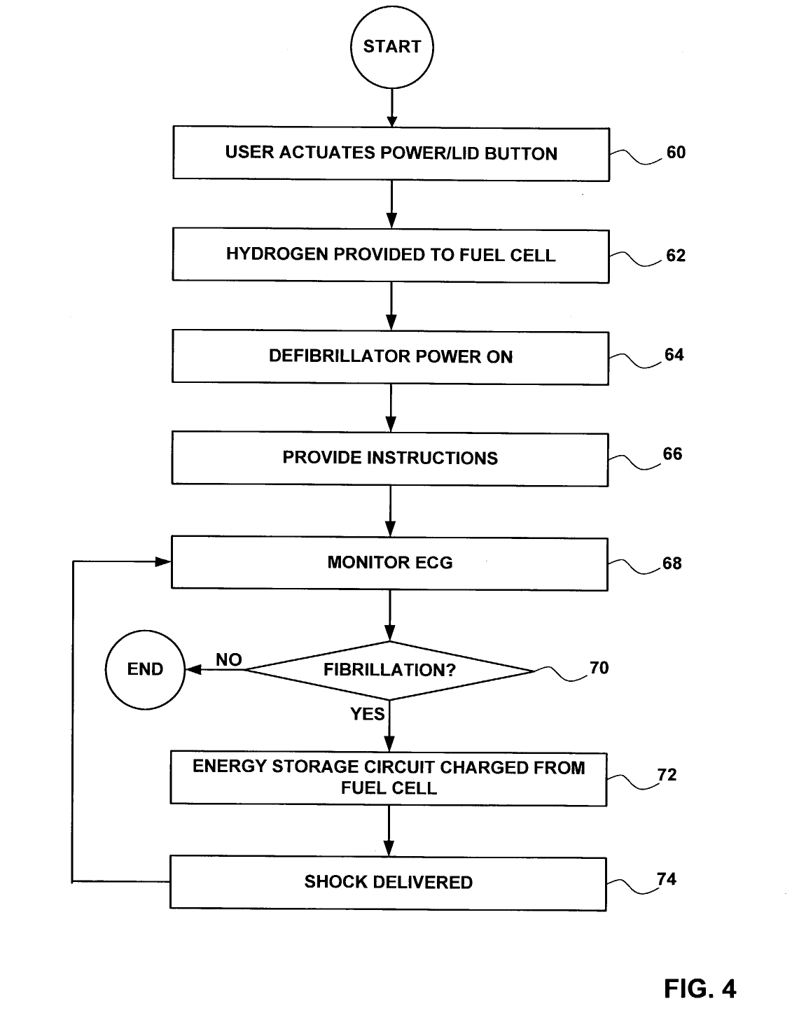

FIG. 4 is a flowchart illustrating an example operation of external defibrillator 10 according to an embodiment of the invention. In particular, FIG. 4 illustrates an example operation of an AED embodiment of defibrillator 10. When a user deploys defibrillator 10 to treat patient 40, activator 46 activates fuel cell 20, e.g., provides hydrogen to fuel cell 20, to power on defibrillator 10.

For example, the user may actuate button 14 (60), which is coupled to puncture member 30, to cause puncture member 30 to pierce membrane 28 and release fuel from container 22. When fuel is released from container 22, hydrogen is provided to fuel cell 20 (62), either directly, or via reformer 26, as discussed above. When hydrogen is provided to fuel cell 20, defibrillator 10 powers on (64), as discussed above.

When defibrillator 10 powers on, power is provided to processor 50 and user interface 54. Processor 50 displays instructions to the user via user interface 54 (66), and monitors the ECG of patient 40 (68). If processor 50 detects fibrillation based on the ECG (70), processor 50 selects a defibrillation shock energy level from a preprogrammed progression of energy levels stored in a memory. Processor 50 directs charging circuit 52 to charge energy storage circuit 48 to a voltage determined based on the selected energy level, and charging circuit 52 transfers energy provided by fuel cell 20 to energy storage circuit 48 as directed by processor 50 (72). Alternatively, processor may direct charging circuit 52 to begin charging energy storage circuit 48 during monitoring of the ECG of patient 40, and may direct charging circuit 52 to charge or discharge energy storage circuit 48 to the selected voltage level if fibrillation is detected. When energy storage circuit 48 reaches the selected voltage, processor 50 or the user may activate a switch within interface 46 to deliver the defibrillation shock to patient 40 (74). Processor 50 continues to monitor the ECG and direct delivery of defibrillation shocks so long as fibrillation is detected.

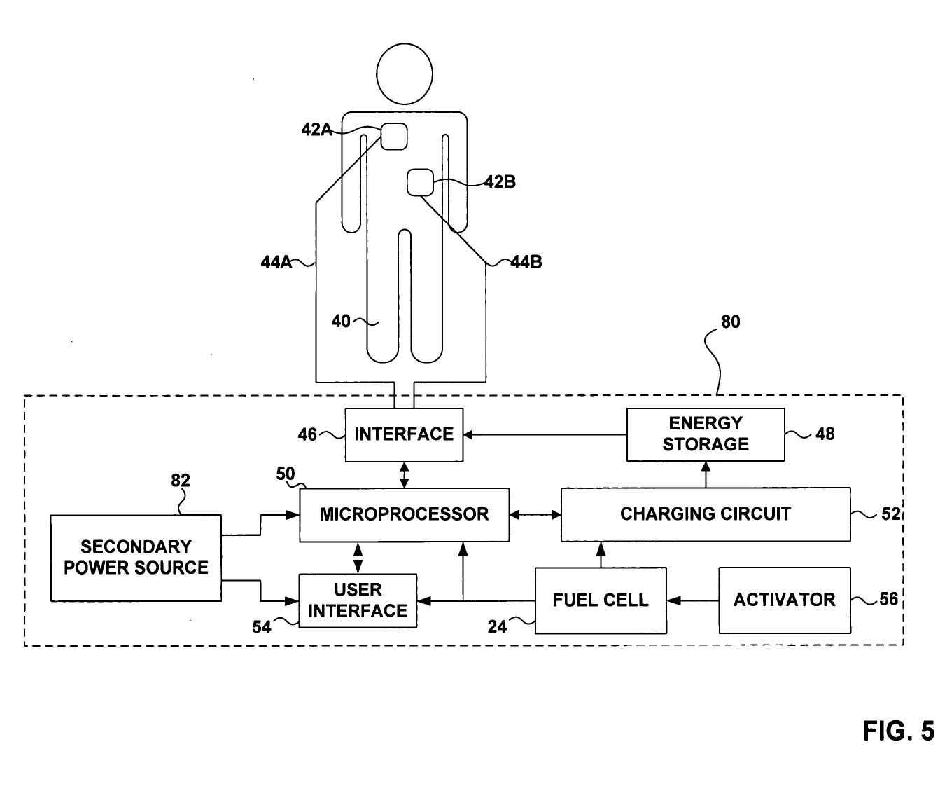

FIG. 5 is a block diagram illustrating components of another example external defibrillator 80. Like defibrillator 10 described above with reference to FIG. 3, defibrillator 80 is coupled to patient 40 by electrodes 42 and conductors 44, and includes an interface 46, an energy storage circuit 48, a processor 50, a charging circuit 52, a user interface 54, an activator 56, and a fuel cell 20. Additionally, defibrillator 80 includes a secondary power source 82, which may be a battery or a second fuel cell.

Secondary power source 82 provides power to components of defibrillator 80 when defibrillator 80 is not in use, i.e., when fuel cell 20 is not activated. For example, secondary power source 82 may, as shown in FIG. 5, provide power to processor 50 and user interface 54 when defibrillator 80 is not in use. By providing power to processor 50 and user interface 54, secondary power source 82 may allow processor 50 to perform self-test routines, and indicate to users the readiness of defibrillator 80 to provide defibrillation therapy via user interface 54, while fuel cell 20 is inactive. In this manner, fuel cell 20 need not be activated until needed to charge energy storage device 48 for delivery of therapy. In some embodiments, secondary power source 82 comprises a rechargeable battery that is recharged by fuel cell 20 when fuel cell 20 is activated.

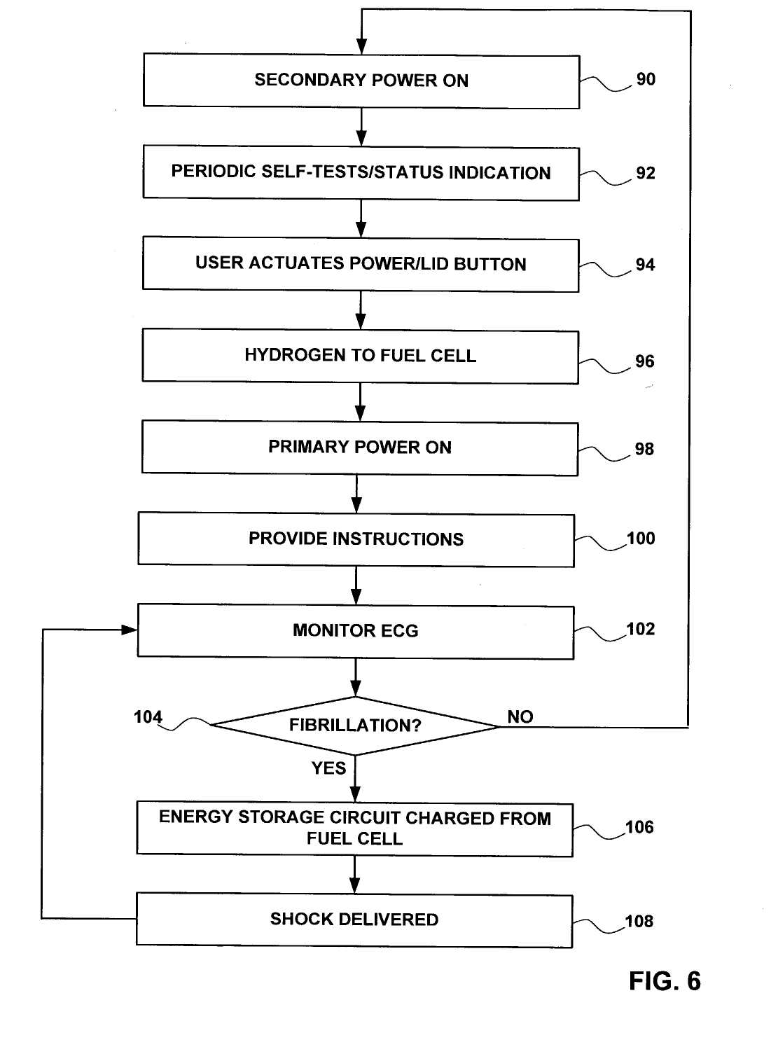

FIG. 6 is a flowchart illustrating an example operation of an AED embodiment of external defibrillator 80 that includes secondary power source 82 according to an embodiment of the invention. During periods when defibrillator 80 is not in use, secondary power source 82 is on (90). With secondary power source 82 on, processor 50 performs periodic self-test routines, and indicates status via user interface 54 (92).

User may actuate button 14 (94) to provide hydrogen to fuel cell 20 (96), as discussed above, to activate fuel cell 20, i.e., turn the primary power for defibrillator on (98). Processor 50 displays instructions to the user via user interface 54 (100), and monitors the ECG of patient 40 (102), as discussed above. If processor 50 detects fibrillation based on the ECG (104), processor 50 selects a defibrillation shock energy level from a preprogrammed progression of energy levels stored in a memory. Processor 50 directs charging circuit 52 to charge energy storage circuit 48 to a voltage determined based on the selected energy level, and charging circuit 52 transfers energy provided by fuel cell 20 to energy storage circuit 48 as directed by processor 50 (106). When energy storage circuit 48 reaches the selected voltage, processor 50 or the user may activate switch 46 to deliver the defibrillation shock to patient 40 (108). Processor 50 continues to monitor the ECG and direct delivery of defibrillation shocks so long as fibrillation is detected.

A number of embodiments of the invention have been described. However, one skilled in the art will appreciate that the invention can be practiced with embodiments other than those disclosed. For example, the invention is not limited to fuel cells that remain inactive until activated by a user. A fuel cell may be activated by a manufacturer of defibrillator 10 prior to delivery of defibrillator 10 to a user. In such embodiments, the fuel cell may remain activated, so long as fuel is provided to the fuel cell, substantially throughout the serviceable life of defibrillator 10. The disclosed embodiments are presented for purposes of illustration and not limitation, and the invention is limited only by the claims that follow.

1. An external defibrillator comprising:
an energy storage circuit to store energy for delivery to a patient as a defibrillation shock;
a fuel cell coupled to the energy storage circuit to provide energy to charge the energy storage circuit for delivery of the defibrillation shock; and
electrodes selectively coupled to the energy storage circuit by a switch to deliver the defibrillation shock to the patient.

2. The external defibrillator of claim 1, further comprising a charging circuit, coupled to the fuel cell and the energy storage circuit, that receives energy from the fuel cell and charges the energy storage circuit with the energy.

3. The external defibrillator of claim 1, further comprising a processor to control operation of the defibrillator, wherein the fuel cell provides energy to power the processor.

4. The external defibrillator of claim 1, further comprising a user interface, wherein the fuel cell provides energy to power the user interface.

5. The external defibrillator of claim 1, further comprising an activator to allow a user to activate the fuel cell.

6. The external defibrillator of claim 5, wherein the activator includes a button, and the user presses the button to activate the fuel cell.

7. The external defibrillator of claim 5, further comprising a container to store a fuel, wherein the activator enables delivery of the fuel from the container to the fuel cell.

8. The external defibrillator of claim 7, wherein the fuel comprises at least one of hydrogen, alcohol, methanol, propane, and butane.

9. The external defibrillator of claim 7, further comprising a reformer to extract hydrogen from the fuel and deliver the hydrogen to the fuel cell, wherein the activator enables delivery of the fuel to the reformer.

10. The external defibrillator of claim 7, wherein the container is at least one of removable, replaceable and refillable to enable the user to refuel the defibrillator.

11. The external defibrillator of claim 1, wherein the energy storage circuit comprises a capacitor.

12. The external defibrillator of claim 1, wherein the defibrillator comprises an automatic external defibrillator.

13. The external defibrillator of claim 1, further comprising:
a processor;
a user interface;
an activator to allow a user to activate the fuel cell; and
a secondary power source to power the processor and the user interface when the fuel cell is not activated.

14. The external defibrillator of claim 13, wherein the processor performs a self-test during a period when the fuel cell is not activated to evaluate readiness of the defibrillator to deliver therapy, and provides an indication of readiness to a user via the user interface.

15. The external defibrillator of claim 13, wherein the fuel cell comprises a first fuel cell, and
wherein the secondary power source comprises a second fuel cell.

16. The external defibrillator of claim 13, wherein the secondary power source comprises a battery.

17. An external defibrillator comprising:
an energy storage circuit to store energy for delivery to a patient as a defibrillation shock;
a fuel cell coupled to the energy storage circuit to provide energy to charge the energy storage circuit for delivery of the defibrillation shock;
electrodes selectively coupled to the energy storage circuit by a switch to deliver the defibrillation shock to the patient; and
an activator to allow a user to activate the fuel cell.

18. The external defibrillator of claim 17, wherein the activator includes a button, and the user presses the button to activate the fuel cell.

19. The external defibrillator of claim 18, further comprising a cover, wherein the user presses the button to open the cover.

20. The external defibrillator of claim 17, further comprising a cover, wherein the activator is coupled to the cover and the user opens the cover to activate the fuel cell.

21. The external defibrillator of claim 17, wherein the activator includes a button, and removal of the defibrillator from a base actuates the button to activate the fuel cell.

22. The external defibrillator of claim 17, further comprising a container to store a fuel, wherein the activator enables delivery of the fuel to the fuel cell.

23. The external defibrillator of claim 22, wherein the fuel comprises at least one of hydrogen, alcohol, methanol, propane, and butane.

24. The external defibrillator of claim 22, further comprising a reformer to extract hydrogen from the fuel and deliver the hydrogen to the fuel cell, wherein the activator enables delivery of the fuel to the reformer.

25. The external defibrillator of claim 22, wherein the container is at least one of removable, replaceable and refillable to enable the user to refuel the defibrillator.

26. The external defibrillator of claim 22, wherein the activator includes a puncture member, the container includes a membrane, and the user actuates the puncture member to puncture the membrane to deliver the fuel to the reformer.

27. An external defibrillator comprising:
a processor;
a user interface;
an energy storage circuit to store energy for delivery to a patient as a defibrillation shock;
a fuel cell coupled to the energy storage circuit to power the processor and the user interface, and to provide energy to charge the energy storage circuit for delivery of the defibrillation shock;
electrodes selectively coupled to the energy storage circuit by a switch to deliver the defibrillation shock to the patient;
an activator to allow a user to activate the fuel cell; and
a secondary power source to power the processor and the user interface when the fuel cell is not activated.

28. The external defibrillator of claim 27, wherein the processor performs a self-test during a period when the fuel cell is not activated to evaluate readiness of the defibrillator to deliver therapy, and provides an indication of readiness to a user via the user interface.

29. The external defibrillator of claim 27, wherein the fuel cell comprises a first fuel cell, and
wherein the secondary power source comprises a second fuel cell.

30. The external defibrillator of claim 27, wherein the secondary power source comprises a battery.

31. The external defibrillator of claim 30, wherein the battery comprises a rechargeable battery.

32. The external defibrillator of claim 31, wherein the fuel cell recharges the battery when activated.

33. The external defibrillator of claim 27, wherein the activator includes a button, and the user presses the button to activate the fuel cell.

34. The external defibrillator of claim 27, wherein the activator enables delivery of fuel to the fuel cell.

35. A method of powering an external defibrillator comprising delivering energy from a fuel cell to components of the defibrillator.

36. The method of claim 35, wherein delivering energy comprises delivering energy to at least one of a processor and a user interface.

37. The method of claim 35, wherein delivering energy comprises delivering energy from the fuel cell to an energy storage circuit, the energy storage circuit storing the energy for delivery to a patient as a defibrillation shock.

38. The method of claim 35, further comprising delivering fuel to the fuel cell, wherein delivering energy comprises delivering energy from the fuel cell as a function of the delivery of fuel to the fuel cell.

39. The method of claim 38, wherein the fuel comprises at least one of hydrogen, alcohol, methanol, propane, and butane.

40. The method of claim 38, wherein delivering fuel comprises delivering fuel to a reformer that extracts hydrogen from the fuel and delivers the hydrogen to the fuel cell.

41. The method of claim 35, wherein delivering energy comprises actuating an activator to cause delivery of energy from fuel cell to the components.

42. The method of claim 41, wherein actuating an activator comprises at least one of pressing a button, opening a lid of the defibrillator, and removing the defibrillator from a base.

43. The method of claim 35, further comprising delivering energy from a secondary fuel source to at least one of the components of the defibrillator when the fuel cell is not activated.

44. A method of operating a defibrillator comprising actuating an activator to activate a fuel cell and power on the defibrillator.

45. The method of claim 44, wherein actuating an activator comprises pressing a button of the defibrillator.

46. The method of claim 44, wherein actuating an activator comprises opening a lid of the defibrillator.

47. The method of claim 44, wherein actuating an activator comprises removing the defibrillator from a base.

48. The method of claim 44, further comprising:
placing electrodes on a patient;
detecting fibrillation of a heart of the patient based on an indication received from the defibrillator; and
directing the defibrillator to deliver a defibrillator shock to the patient based on the detection.
